(19) United States
(12) Patent Application Publication
(10) Pub. No.: US 2019/0298307 A1
(43) Pub. Date: Oct. 3, 2019

(54) EXPANSION MODULE FOR ULTRASOUND SYSTEM

(71) Applicant: Qisda Corporation, Taoyuan City (TW)

(72) Inventor: Chia-En CHUANG, Hsinchu County (TW)

(73) Assignee: Qisda Corporation, Taoyuan City (TW)

(21) Appl. No.: 16/357,420

(22) Filed: Mar. 19, 2019

(30) Foreign Application Priority Data
Mar. 27, 2018 (CN) 201810259394.0

(51) Int. Cl. A61B 8/00; H01R 29/00; H01R 31/06

(52) U.S. Cl. CPC A61B 8/4411; A61B 8/4433; H01R 2201/20; H01R 31/065; H01R 29/00

(57) ABSTRACT

The present invention discloses an expansion module connected to an ultrasound host. The expansion module includes a case, a first plug, a second plug, multiple probe slots, a path switching unit, and a control unit. The first plug is disposed on a first side of the case. The second plug is disposed on a second side of the case and matched with the first plug. The multiple probe slots are disposed on a third side of the case and coupled to one or more probes. The path switching unit is coupled to the first plug and probe slots. The control unit is coupled to the first plug, the second plug, and the path switching unit.

This application claims the benefit of People's Republic of China application Serial No. 201810259394.0, filed Mar. 27, 2018, the subject matter of which is incorporated herein by reference.

### BACKGROUND OF THE INVENTION

### Field of the Invention

The invention relates in general to an expansion module, and more particularly to an expansion module used in an ultrasound system.

### Description of the Related Art

Ultrasound is sound waves or vibrations with frequencies higher than the upper audible limit of human hearing, being 20 kHz. Ultrasound has been widely used in various fields, including radar technology for military purpose, deep sea exploration for exploration purpose, and organ detection for medical purpose.

In terms of the medical purpose, the ultrasound system or apparatus normally includes an ultrasound host in conjunction with one or more probes for performing ultrasound detection. The user can use corresponding probes to detect a particular area, organ or range. However, due to hardware restrictions, the quantity of the probe slots configured in the ultrasound host is limited. Once the needed quantity of probes or types is over the existing quantity of probe slots, the user will have to unplug some of the probes already plugged to the probe slots before he/she can plug any additional probes to the fully occupied probe slots.

### SUMMARY OF THE INVENTION

To resolve the above problems, the present invention discloses an expansion module, particularly to an expansion module used in an ultrasound system or apparatus to provide more convenience of use to the user.

According to one embodiment of the present invention, an expansion module connected to an ultrasound host is disclosed. The expansion module includes a case, a first plug, a second plug, multiple probe slots, a path switching unit and a control unit. The first plug is disposed on a first side of the case. The second plug is disposed on a second side of the case and matched with the first plug. The multiple probe slots are disposed on a third side of the case and coupled to one or more probes. The path switching unit is coupled to the first plug and probe slots. The control unit is coupled to the first plug, the second plug, and the path switching unit.

In another embodiment, the above expansion module is used in an ultrasound system, the first plug is a male plug, and the second plug is a female plug.

The expansion module of the present invention can increase the quantity of probes that can be connected to the ultrasound host. For the user, the ultrasound system is more convenient to use, and the overall ultrasound detection time can be reduced.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 is a schematic diagram of an expansion module according to an embodiment of the present invention;

FIG. 2 is a schematic diagram of an ultrasound system according to an embodiment of the present invention;

FIG. 3 is a schematic diagram of an expansion module according to another embodiment of the present invention; and

FIG. 4 is a schematic diagram of an ultrasound system according to another embodiment of the present invention.

### DETAILED DESCRIPTION OF THE INVENTION

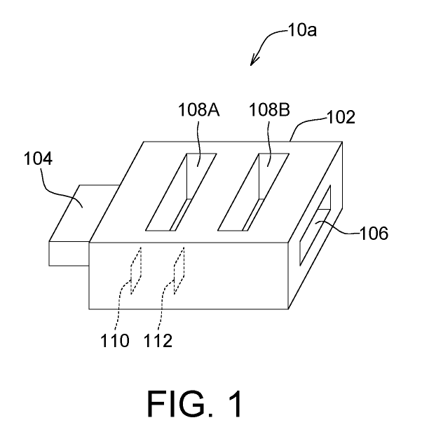

Referring to FIG. 1, a schematic diagram of an expansion module according to an embodiment of the present invention is shown. The expansion module 10a can be connected to an ultrasound host (not illustrated). The expansion module 10a includes a case 102, a first plug 104, a second plug 106, multiple probe slots 108A and 108B, a path switching unit 110, and a control unit 112.

The first plug 104 can be disposed on a first side of the case 102. The second plug 106 can be disposed on a second side of the case 102, wherein the second side is different from the first side. In the present embodiment, the second side and the first side are opposite to each other, but the present invention is not limited thereto. For example, the first side and the second side can be adjacent to each other or disposed on the same side. The second plug 106 is matched with the first plug 104. For example, the first plug 104 can be a male plug, the second plug 106 can be a female plug matched with the first plug 104. The probe slots 108A and 1086 are disposed on a third side of the case 102, wherein the third side is different from the first side and the second side. The probe slots 108A and 108B can respectively be coupled to an independent probe. One or more probe slots (not illustrated) can be disposed on the third side or other side of the case 102, but the quantity of probe slots is not specified here.

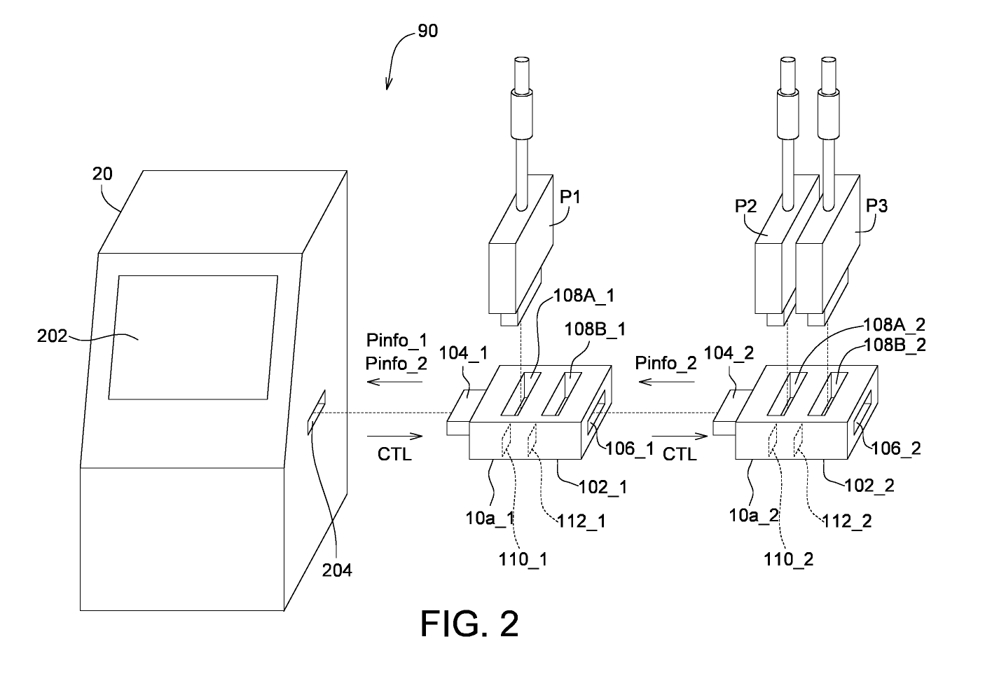

The path switching unit 110 is coupled to the first plug 104 and the probe slots 108A and 108B. The control unit 112 is coupled to the first plug 104, the second plug 106, and the path switching unit 110. In an embodiment, the path switching unit 110 and the control unit 112 can be disposed inside the case 102. While the structure of the expansion module 10a is as disclosed above, the operation of the expansion module 10a will be disclosed below with the ultrasound system as indicated in FIG. 2.

Referring to FIG. 2, a schematic diagram of an ultrasound system according to an embodiment of the present invention is shown. The ultrasound system 90 includes an ultrasound host 20 and one or more the expansion modules 10a_1 and 10a_2 (the quantity of expansion modules is exemplified by two in the the present embodiment). The expansion modules 10a_1 and 10a_2 can be realized by the expansion module 10a of FIG. 1, and have the same structure and function.

The ultrasound host 20 may include a user interface 202 and one or more the host slot 204. The first plugs 104_1 and 104_2 of the expansion modules 10a_1 and 10a_2 can be matched with the host slot 204. Therefore, in terms of the ultrasound system 90, the expansion module 10a_1 can be connected to the ultrasound host 20 when the first plug 104_1 is plugged to the host slot 204; the expansion module 10a_2 can be connected to the expansion module 10a_1 when the first plug 104_2 is plugged to the second plug 106_1 of the expansion module 10a_1.

One or more probes P1-P3 can be selectively plugged to the probe slots 108A_1 and 108B_1, 108B_1, 108B_2 of the expansion modules 10a_1 and 10a_2. For example, the probe P1 is plugged to the probe slot 108A_1 of the expansion module 10a_1; probe P2 is plugged to the probe slot 108A_2 of the expansion module 10a_2; and probe P3 is plugged to the probe slot 108B_2 of the expansion module 10a_2. The control unit 112_1 of the expansion module 10a_1 can detect whether any probe is plugged to the probe slots 108A_1 and 108B_1. If yes, the control unit 112_1 will obtain a corresponding probe information, which may include such as the numbering of the probe slot and the model of the probe plugged to the probe slot. For example, the probe information obtained by the control unit 112_1 of the expansion module 10a_1 may include that the probe slot 108A_1 (the numbering of the probe slot) has a probe P1 (the model of the probe) plugged therein, but the probe slot 108B_1 does not have any probe plugged therein. Similarly, the control unit 112_2 of the expansion module 10a_2 will perform the same operation. After the expansion module 10a_1 obtains the probe information Pinfo_1, the control unit 112_1 of the expansion module 10a_1 can transmit probe information Pinfo_1 to the ultrasound host 20 through the first plug 104_1. After the expansion module 10a_2 obtains the probe information Pinfo_2, the control unit 112_2 of the expansion module 10a_2 can transmit the probe information Pinfo_2 to the expansion modules 10a_1 through the first plug 104_2 and further transmit the probe information Pinfo_2 to the ultrasound host 20 through the expansion module 10a_1.

After receiving probe information Pinfo_1 and Pinfo_2 from the expansion modules 10a_1 and 10a_2, the ultrasound host 20 display corresponding message on the user interface 202 according to the received probe information Pinfo_1 and Pinfo_2. The user can select one of the probes to use through the user interface 202. The ultrasound host 20 generates a control signal CTL according to the user's selection. The control unit 112_1 of the expansion module 10a_1 can receive the control signal CTL from the ultrasound host 20 through the first plug 104_1, and further operate the path switching unit 110_1 to select at most one of the probe slots 108A_1 and 108A_2 according to the control signal CTL. The control unit 112_1 of the expansion module 10a_1 further transmits the control signal CTL to the next stage of expansion module (that is, the expansion module 10a_2) through the second plug 106_1. Similarly, the expansion module 10a_2 will perform the same operation.

For example, when the user selects the probe P1 through the user interface 202, the control unit 112_1 of the expansion module 10a_1 will operate the path switching unit 110_1 to select the probe slots 108A_1 but not the probe slot 108B_1 according to the control signal CTL, and the control unit 112_2 of the expansion module 10a_2 will operate the path switching unit 110_2 not to select the probe slots 108A_2 and 108B_2 according to the control signal CTL. Through the above operation, a signal transmission path can be established between the probe P1 and the ultrasound host 20. In other words, the detection information obtained through the selected probe P1 can be transmitted to the ultrasound host 20 via the first plug 104_1 of the expansion module 10a_1 and the host slot 204 of the ultrasound host 20.

For example, when the user selects the probe P2 through the user interface 202, the control unit 112_1 of the expansion module 10a_1 will operate the path switching unit 110_1 not to select the probe slots 108A_1 and 108B_1 according to the control signal CTL, and the control unit 112_2 of the expansion module 10a_2 will operate the path switching unit 110_2 to select the probe slot 108A_2 but not the probe slot 108B_2 according to the control signal CTL. Through the above operation, a signal transmission path can be established between the probe P2 and the ultrasound host 20. In other words, the detection information obtained through the selected probe P2 can be transmitted to the ultrasound host 20 via the probe slots 108A_2 of the expansion module 10a_2, the first plug 104_2, the second plug 106_1 of the expansion module 10a_1, the first plug 104_1 and the host slot 204 of the ultrasound host 20.

In some embodiments, the expansion module 10a_1 can be connected to the ultrasound host 20 through an adaptor. The adaptor may include a first adaptor plug and a second adaptor plug, wherein the first adaptor plug is matched with the host slot 204 of the ultrasound host 20, the second adaptor plug is matched with the first plugs 104_1 and 104_2 of the expansion modules 10a_1 and 10a_2.

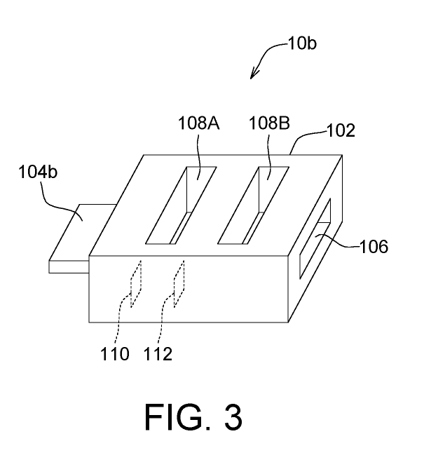

In some embodiments, the first plugs 104_1 and 104_2 of the expansion modules 10a_1 and 10a_2 are not matched with the host slot 204 of the ultrasound host 20. The expansion module 10a can be connected to the ultrasound host 20 through a first expansion module 10b. As indicated in FIG. 3, the structure of the first expansion module 10b is similar to that of the expansion module 10a, and function and operation of the first expansion module 10b are the same as that of the expansion module 10a except that the first plug 104b of the first expansion module 10b are not matched with the second plug 106 but are matched with the host slot 204 of the ultrasound host 20.

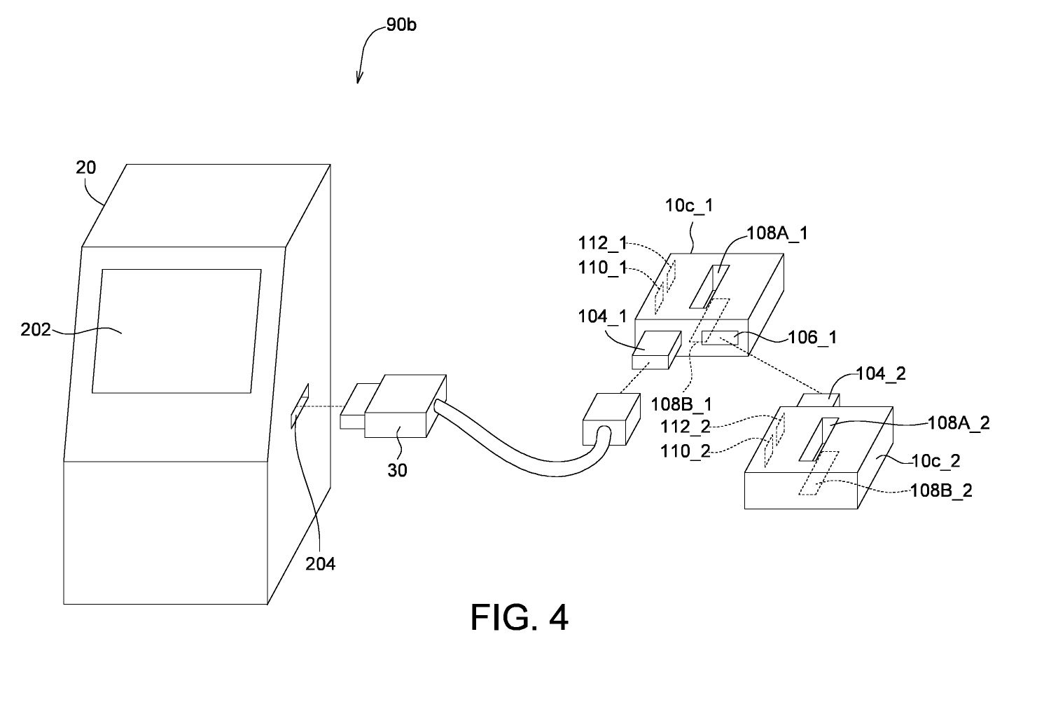

Referring to FIG. 4, a schematic diagram of an ultrasound system according to another embodiment of the present invention is shown. In terms of the ultrasound system 90b, the structure of the expansion modules 10c_1 and 10c_2 is only slightly different from that of the expansion module 10a. The expansion module 10c_1 is only different from the expansion module 10a in that the first plug 104_1 and the second plug 106_1 are disposed on a first side of the case 102, and the probe slot 108A_1 and the probe slot 108B_1 are respectively disposed on a second side and a third side of the case 102 (the third side is opposite to the second side and is not illustrated). The expansion module 10c_2 is different from the expansion module 10a in a similar way. In the present embodiment, the expansion module 10c_1 is connected to the ultrasound host 20 through an adaptor 30. The above mentioned features and function of the ultrasound system disclosed in previous embodiment are the same as that disclosed in the present embodiment and not repeated here.

In the above embodiments, the quantity of the expansion modules plugged to the ultrasound host is exemplified by two. In actual application, the quantity of expansion modules plugged to corresponding host slots is determined according to actual needs. For example, when the ultrasound host has only two host slots but the user needs to use seven probes, the user plug three expansion module connected in series to the ultrasound host to achieve the expansion effect.

As disclosed above, since the expansion module of the present invention can increase the quantity of probes connected to the ultrasound host, the user, instead of unplugging any probe from a host slot, only needs to select a probe through the user interface, and the ultrasound host will then establish a signal transmission path with the selected probe. For the user, the ultrasound system is more convenient to use, and the overall ultrasound detection time can be reduced.

While the invention has been described by way of example and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An expansion module connected to an ultrasound host, the expansion module comprising:
a case;
a first plug disposed on a first side of the case;
a second plug disposed on a second side of the case and matched with the first plug;
a plurality of probe slots disposed on a third side of the case and coupled to one or more probes;
a path switching unit coupled to the first plug and the probe slots; and
a control unit coupled to the first plug, the second plug, and the path switching unit.

2. The expansion module according to claim 1, wherein, when a probe is selectively plugged to the probe slots, the control unit obtains a probe information of the probe by way of detection and further transmits the probe information to the ultrasound host through the first plug.

3. The expansion module according to claim 1, wherein the control unit receives a control signal from the ultrasound host through the first plug and further operates the path switching unit according to the control signal to select at most one of the probe slots, and the ultrasound host establishes a signal transmission path with the selected probe slot.

4. The expansion module according to claim 1, wherein the expansion module can be connected to a first expansion module having the same composition with the expansion module, the expansion module is connected to a first plug of the first expansion module via the second plug of the expansion module, and the control unit of the expansion module receives one or more probe information through the second plug of the expansion module and further transmits the one or more probe information to the ultrasound host through the first plug of the expansion module.

5. The expansion module according to claim 4, wherein the control unit of the expansion module receives a control signal from the ultrasound host through the first plug of the expansion module and further transmits the control signal to the first expansion module through the second plug of the expansion module, and the ultrasound host establishes a signal transmission path with a probe slot of the first expansion module.

6. The expansion module according to claim 1, wherein the expansion module can be connected to a first expansion module having the same composition with the expansion module, the expansion module is connected to a second plug of the first expansion module via the first plug, and a first plug of the first expansion module is connected to the ultrasound host.

7. The expansion module according to claim 6, wherein, when a probe is selectively plugged to the probe slots of the expansion module, the control unit of the expansion module obtains a probe information of the probe by way of detection and further transmits the probe information to the ultrasound host through the first plug of the expansion module and the first expansion module.

8. The expansion module according to claim 6, wherein the control unit of the expansion module receives a control signal of the ultrasound host from the first expansion module through the first plug of the expansion module and further operates the path switching unit of the expansion module according to the control signal to select at most one of the probe slots, and the ultrasound host establishes a signal transmission path with the selected probe slot.

9. The expansion module according to claim 1, wherein the first plug is connected a host slot of an ultrasound host directly or through an adaptor.

10. An expansion module ultrasound system as disclosed in claim 1, wherein when the first plug is a male plug, the second plug is a female plug; when the first plug is a female plug, the second plug is a male plug.

11. An ultrasound system comprising:
an ultrasound host; and
a expansion module connected to the ultrasound host, the expansion module comprising:
a case;
a first plug disposed on a first side of the case;
a second plug disposed on a second side of the case and matched with the first plug;
a plurality of probe slots disposed on a third side of the case and coupled to one or more probes;
a path switching unit coupled to the first plug and the probe slots; and
a control unit coupled to the first plug, the second plug, and the path switching unit.

12. The ultrasound system according to claim 11, wherein, when a probe is selectively plugged to the probe slots, the control unit obtains a probe information of the probe by way of detection and further transmits the probe information to the ultrasound host through the first plug.

13. The ultrasound system according to claim 11, wherein the control unit receives a control signal from the ultrasound host through the first plug and further operates the path switching unit according to the control signal to select at most one of the probe slots, and the ultrasound host establishes a signal transmission path with the selected probe slot.

14. The ultrasound system according to claim 11, further comprising:
a first expansion module having the same composition with the expansion module, wherein the expansion module is connected to a first plug of the first expansion module via the second plug of the expansion module, and the control unit of the expansion module receives one or more probe information through the second plug of the expansion module and further transmits the one or more probe information to the ultrasound host through the first plug of the expansion module.

15. The ultrasound system according to claim 14, wherein the control unit of the expansion module receives a control signal from the ultrasound host through the first plug of the expansion module and further transmits the control signal to the first expansion module through the second plug of the expansion module, and the ultrasound host establishes a signal transmission path with a probe slot of the first expansion module.

16. The ultrasound system according to claim 11, further comprising:
a first expansion module having the same composition with the expansion module, wherein the expansion module is connected to a second plug of the first expansion module via the first plug of the expansion module, and a first plug of the first expansion module is connected to the ultrasound host.

17. The ultrasound system according to claim 16, wherein, when a probe is selectively plugged to the probe slots of the expansion module, the control unit of the expansion module obtains a probe information of the probe by way of detection and further transmits the probe information to the ultrasound host through the first plug of the expansion module and the first expansion module.

18. The ultrasound system according to claim 16, wherein the control unit of the expansion module receives a control signal of the ultrasound host from the first expansion module through the first plug of the expansion module and further operates the path switching unit of the expansion module according to the control signal to select at most one of the probe slots, and the ultrasound host establishes a signal transmission path with the selected probe slot.

19. The ultrasound system according to claim 11, wherein the first plug of the expansion module is connected a host slot of an ultrasound host directly or through an adaptor.

20. An ultrasound system ultrasound system as disclosed in claim 11, wherein when the first plug is a male plug, the second plug is a female plug; when the first plug is a female plug, the second plug is a male plug.
